(19) United States
(12) Patent Application Publication
(10) Pub. No.: US 2026/0151176 A1
(43) Pub. Date: Jun. 4, 2026

(54) HF-GENERATOR FOR SUPPLYING ONE OR MORE MEDICAL INSTRUMENTS

(71) Applicant: Erbe Elektromedizin GmbH, Tuebingen (DE)

(72) Inventors: Falk HATZFELD, Tuebingen (DE); Marc KEGREISS, Rottenburg (DE); Michael SAUTER, Albstadt (DE)

(21) Appl. No.: 19/400,002

(22) Filed: Nov. 25, 2025

(30) Foreign Application Priority Data
Dec. 3, 2024 (EP) 24217261.7

(51) Int. Cl. A61B 18/12; A61B 18/00; H02N 11/00

(52) U.S. Cl. CPC A61B 18/1206; H02N 11/002; A61B 2018/00702; A61B 2018/00827

(57) ABSTRACT

Disclosed are HF generators for the supply of at least one medical instrument. Disclosed HF generators include an output block, an oscillator block, a communication block and a grid block. The output block is configured to supply the one or more medical instruments with an HF current. The oscillator block is configured to supply the output block via a first power coupler with the HF current. In one example a first power coupler and a first data coupler comprise a higher insulation voltage than the remaining power couplers and the data coupler, where the output block is protected in relation to oscillator block by means of only two couplers, which are provided with measures for increasing their insulation voltage, while providing similar functionality and equal dielectric strength.

### CROSS-REFERENCE TO RELATED APPLICATIONS

This application claims priority to European Patent Application No. 24217261.7, filed Dec. 3, 2024, the entirety of which is incorporated herein.

The invention refers to a high frequency generator (HF generator) for supply of at least one medical instrument, particularly of at least one HF surgical instrument, that is for example configured for cutting, coagulating as well as for achieving additional tissue effects of biological tissue of a human or animal patient as desired.

HF generators for supply of one or more medical instruments are known from the prior art in general.

WO 2004/030552 A1 describes an electrosurgical generator for the supply of one or more medical apparatuses. The medical apparatuses shall allow an influence of biological tissue of a patient, such as cutting or coagulating of tissue. For this purpose, the medical apparatuses are at least temporarily attached on the patient or get into contact with the patient. Therefore, it is particularly important to guarantee that no uncontrolled current can flow from the medical apparatuses via the patient. The patient, who is capacitively coupled against ground in relation to the current supply grid shall be maintained isolated. In order to guarantee this WO 2004/030552 A1 comprises a multiplicity of insulation locations between the supply grid, the control unit and the oscillator of the generator respectively, that are configured with increased electrical strength. The measures for achieving an increased electrical strength of the individual insulation locations are complex with regard to the construction. A multiplicity of such insulation locations therefore results in a comparably high complexity of the system architecture.

Moreover, in case of multiple insulation locations arranged in parallel, that have unequal dielectric strengths for manufacturing reasons, for example, the insulation location having the lowest dielectric strength is the weak point of the system architecture, so that the system safety can be partly regarded as being critical in spite of a comparably complex system architecture.

### BRIEF SUMMARY

Starting therefrom, the object of the present invention is to provide an HF generator having an improved and simplified isolation scheme. Particularly, in the HF generator, less or at least less particularly dielectrically strong insulation locations shall be required-the latter with at least equal or improved safety.

This object is solved by means of an HF generator according to the disclosed embodiments and the claims.

The HF generator according to the invention serves for supply of at least one medical instrument, which is particularly at least one HF surgical instrument. The HF generator according to the invention comprises an output block, an oscillator block, a communication block and a grid block.

The output block is configured to supply the one or the multiple medical instruments with HF current. HF currents are thereby alternating currents that can have a frequency of above 100 kHz, preferably above 300 kHz, for example between 300 kHz and 4 MHz. The output block particularly comprises a sensor unit that is configured to determine sensor data of the HF currents, such as current values, voltage values, apparent power values, active power values and/or reactive power values, but also values such as the complex impedance of the instrument or the tissue, changes thereof and/or values for the linearity of the complex impedance. The output block can additionally comprise a preprocessing unit that is configured to preprocess the sensor data, preferably in real time.

The oscillator block is configured to supply the output block with the HF current via a first power coupler. In addition, the oscillator block is connected via a first data coupler with the output block. The oscillator block particularly comprises an HF control unit that is configured to receive the desired parameters of the HF currents and the sensor data of the output block and to control the HF currents based on the parameters and the sensor data in closed-loop manner.

The communication block is connected with the out put block via a second power coupler and with the oscillator block via a second data coupler. The grid block is coupled with the oscillator block and via a third power coupler with the communication block in a supplying manner.

A particularity of the HF generator according to the invention is that the first power coupler comprises a higher insulation voltage than the remaining power couplers, preferably all remaining power couplers. Preferably also the first data coupler comprises a higher insulation voltage than the remaining data couplers, preferably all remaining data couplers. The higher insulation voltages of the first power coupler and the first data coupler mean that the latter have a higher dielectrical strength with regard to voltage peaks and potential differences between the grid, the HF generator and the patient.

The insulation concept according to the invention provides a clear isolation between the output block and the oscillator block in order to decrease the number of insulation locations, particularly the number of insulation locations having high dielectric strength. The oscillator block is only sparsely protected or not protected at all relative to the grid block, while the output block is strongly protected relative to the oscillator block-that means, the insulation locations between the output block and the oscillator block comprise a high insulation voltage or insulation strength.

In doing so, the output block can be insulated with only two couplers of high dielectrical strength—a (first) power coupler and a (first) data coupler—relative to the oscillator block providing an increased degree of dielectric strength. The isolation scheme of the HF generator can be remarkably simplified in this manner. The input and the output of the first power coupler and the first data coupler are galvanically isolated from each other with high dielectric strength in each case. The dielectric strength of a coupler is thereby defined by the maximum insulation voltage or breakdown voltage that can apply between its primary side and its secondary side without resulting in a voltage breakthrough or in a current flow. The first power coupler can be, for example, a transformer having separate windings insulated from one another, which are galvanically and spatially separated from each other. The first power coupler comprises a particularly high electrical insulation strength between primary and secondary side. For example, this can be achieved by arranging the primary and secondary windings in separate encapsulated insulating material chambers of the first power coupler. Due to the reduction down to only one power coupler with increased safety against voltage breakthrough (increased insulation strength), the technical effort for the HF generator can be reduced while providing equal or even increased safety.

Preferably, the isolation voltage to which the first power coupler and the first data coupler are dimensioned is larger than the sum of the two-fold output side peak voltage of the oscillator block and an additional voltage that can be present on the operating voltage of the oscillator block in form of a maximum voltage peak. This maxi mum voltage peak is the sum of the operating direct voltage of the oscillator block and the two-fold grid peak voltage, wherein this sum is additionally multiplied with a safety factor. The safety factor can be, for example, minimum 1, 2, 3, 4 or more. The relatively large dimensioned safety factor reduces the danger that the alternating current of the grid block can break through onto the output block and thus via a connected medical instrument on the patient and/or the surgeon.

For all couplers, it expressly applies: a power coupler is configured to transmit power between two blocks, wherein the two blocks are galvanically insulated from each other. A data coupler is configured to transmit data-that is information-between two blocks, wherein both blocks are also galvanically insulated from one another.

Between the output block and the oscillator block, particularly a first insulation path can be formed in which the first power coupler and the first data coupler are arranged parallel to one another. An insulation path is a conceivable path, which is however interrupted by one or more insulating barriers, along which a current cannot and must not flow from one end to the other end of the insulation path. For this purpose, the one or more barriers present in the insulation path comprise a defined insulation voltage respectively, that determines the insulation voltage of the insulation path. The first power coupler and the first data coupler preferably comprise equal insulation voltages.

Preferably, a second insulation path is additionally formed between the output block and the oscillator block, in which the second power coupler and the second data coupler are arranged in series, so that the insulation voltages of the second power coupler and the second data coupler add to each other along the second insulation path.

The first insulation path and the second insulation path are particularly arranged parallel to one another. The insulation voltages of the first insulation path and the second insulation path are preferably equal.

Particularly, the insulation voltage of the first power coupler and the first data coupler are each higher, preferably considerably higher, than the insulation voltage of the second power coupler and the second data coupler-for example 1.5 times, 2 times or 2.5 times higher.

It is preferred if the summed insulation voltage of the second power coupler and the second data coupler corresponds to the insulation voltage of the first power coupler, preferably coincides therewith. It is likewise preferred if the summed insulation voltage of the second power coupler and the second data coupler corresponds to the insulation voltage of the first data coupler, preferably coincides therewith.

This results in that the first insulation path and the second insulation path comprise equal insulation voltages, whereby due to the parallel arrangement of the first insulation path relative to the second insulation path in case of overvoltage none of the insulation paths yields, but both withstand the voltage peak without voltage breakthrough.

The first and the second data coupler are preferably configured as inductive or capacitive data couplers or as optocouplers. However, the first and second and third power couplers can be configured as a transformer in each case.

Preferably, the oscillator block comprises an HF unit, an HF control unit, a grid unit and a second data unit. The HF control unit is particularly configured to control the HF unit so that the HF unit creates HF currents that are transmitted via the first power coupler to the output block. The HF currents can have different varying HF characteristics, which can be modified during operation of the HF generator using the HF control unit. For example, the HF characteristics can thereby comprise different current values, voltage values, frequency values, waveforms, crest factors, clockings and the like in order to define different operation modes.

The grid unit is particularly configured to supply the HF unit with grid power coming from the grid block. The grid unit can comprise a power factor correction unit in order to make the current drawn on the grid side approximately sinusoidal shaped and to reduce its harmonics. The HF unit can be configured to control the power factor correction unit of the grid unit also in advance as necessary in order to always provide sufficient power, especially in case of abrupt load changes.

The output block preferably comprises a distribution unit. The latter is configured to distribute the HF voltage increased by transformation and received via the first power coupler to the one or more medical instruments.

The HF voltage can have an amount of more than 2 kV, 3 kV or 4 kV, for example.

The distribution unit particularly comprises at least one sensor unit that is configured for detection of the HF currents as sensor data in the output block. For example, the sensor data can comprise at least current values, voltage values, apparent power values, active power values and/or reactive power values.

In addition, the output block can comprise a pre-processing unit that is communicatively coupled with the sensor unit. The preprocessing unit can be only an analog-digital-converter in the simplest case, with which the analog sensor data can be converted into digital sensor data. The preprocessing unit can, however, also be configured to carry out more complex preprocessing steps, such as the determination of the complex tissue impedance, the determination of the change of the complex tissue impedance and/or the determination of a linearity value of the complex tissue impedance. The preprocessing unit is preferably configured to process the sensor data in real time.

The output block can additionally comprise a first data unit that is configured for buffering the (digital) sensor data and for providing the sensor data to the oscillator block. The first data unit is particularly communicatively connected with the second data unit via the first data coupler so that sensor data can be exchanged between the first data unit and the second data unit.

The communication block can comprise an operation control unit and, connected with the control unit, a communication interface toward multiple operation and indication units. Via the operation and indication units the user of the HF generator, that is an operating doctor, a surgeon, an assistant doctor or a surgery assistant, can set desired parameters for the HF control unit.

The operation control unit is communicatively connected with the second data unit via the second data coupler, so that the second data unit can receive the preset parameters for the HF control unit via the second data interface from the operation control unit. In addition, the second data unit can receive the detected sensor data of the output block via the first data coupler and buffer the detected sensor data. The preset parameters and the detected sensor data can be forwarded to the HF control unit that is configured to control the HF unit, so that the HF currents in the output block are provided with the preset HF characteristics (parameters) and are controlled in feedback manner.

Preferably the first power coupler and the first data coupler are assigned to a first insulation class in which the couplers withstand (particularly) high voltages, such as 8 kV, 10 kV, 12 kV or more. The remaining power and data couplers are, however, assigned to second insulation class in which the couplers, in comparison, only withstand lower voltages, such as 4 kV, 5 kV, 6 kV or less-without a voltage breakthrough occurring between the primary side and the secondary side.

The output block can be protected via the second power coupler and the third power coupler relative to the grid block.

Due to the inventive system architecture of the HF generator, the blocks of the HF generator are divided in different insulation zones relative to the grid block, for example in a high insulation zone, an intermediate insulation zone and a low insulation zone. The high insulation zone is best protected against voltage breakthrough owing to a voltage peak resulting from the addition of a grid side voltage peak and a voltage produced by the HF generator. The output block is located in the high insulation zone, the communication block is located in the intermediate insulation zone and the oscillator block is located in the low insulation zone.

Further details of preferred embodiments of the invention are derived from the claims, the drawings, or the description.

### BRIEF DESCRIPTION OF THE SEVERAL VIEWS OF THE DRAWINGS

FIG. 1 an example of the HF generator according to the invention in schematic illustration;

FIG. 2 an insulation diagram of the insulation concept for the HF generator according to the invention;

FIG. 3 another insulation diagram of the insulation concept for the HF generator according to the invention;

FIG. 4 an example of the first data coupler and the first power coupler;

FIG. 5 another example of the first data coupler and the first power coupler;

FIG. 6 an example of the grid unit in the oscillator block; as well as

FIG. 7 an example of the HF unit together with the oscillator block and the connection to the output block.

### DETAILED DESCRIPTION

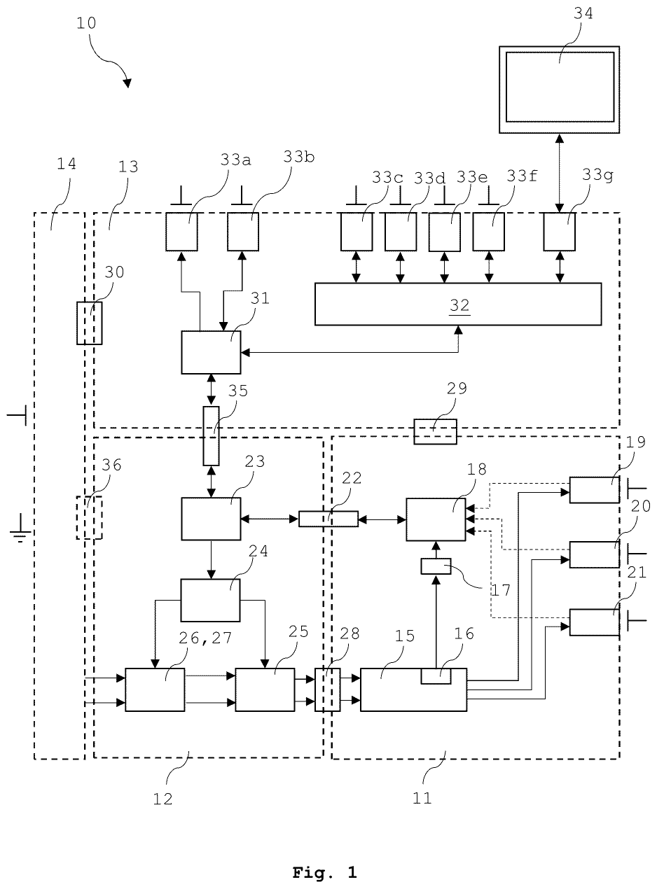

FIG. 1 shows an exemplary illustration of an HF generator 10 according to the invention. The HF generator 10 comprises an output block 11, an oscillator block 12, a communication block 13 and a grid block 14.

The output block 11 serves for supply of the medical instruments with HF currents that are produced in the oscillator block 12. The oscillator block 12 is supplied with grid voltage by means of grid block 14. The communication block 13 serves as an interface to an operator of the HF generator 10 via which the operator can adjust the HF currents produced in the oscillator block 12.

The output block 11 comprises a distribution unit 15 having a sensor unit 16, a preprocessing unit 17, and a first data unit 18. In addition, the output block 11 illustrated in FIG. 1 comprises a first instrument interface 19, a second instrument interface 20 and a neutral electrode interface 21.

The first instrument interface 19 and the second instrument interface 20 are configured for connecting a first and second medical instrument to the output block 11 of the HF generator 10. The neutral electrode interface 21 is configured for connecting a neutral electrode that can be attached to a patient. The medical instruments can be monopolar and/or bipolar HF surgical instruments. The instrument and neutral electrode interfaces 19, 20, 21 are supplied with HF currents by means of the distribution unit 15. The instrument and neutral electrode interfaces 19, 20, 21 can be configured to determine whether a medical instrument is connected and communicate it to the first data unit. The instrument and neutral electrode interfaces 19, 20, 21 can be additionally configured to identify connected instruments.

The sensor unit 16 is configured to detect sensor data of the produced HF currents. For example, such sensor data can comprise current values, voltage values, power values, complex impedances, and the like. The sensor unit 16 is connected with the preprocessing unit 17.

The preprocessing unit 17 can be an analog-digital converter in the simplest case which makes discrete data from the received analog sensor data. The preprocessing unit 17 can, however, also be configured to carry out more complex preprocessing steps, by means of which the sensor data can be preprocessed, as far as possible in real time. For example, sensor value progresses can be smoothened using a moving average filter. Other preprocessings of the sensor data, such as noise suppression, normalization of the data, filtering, and the like are also possible. The preprocessing unit 17 is connected with the first data unit 18 and is configured for forwarding the digital sensor data to the first data unit 18.

Alternatively, the sensor unit 16 can also already comprise an analog-digital converter. In this case, the sensor unit 16 can be directly connected with the first data unit 18. The digital sensor data can be directly forwarded from the sensor unit 16 to the first data unit 18.

The first data unit 18 is configured for buffering the digital sensor data. The first data unit 18 is connected with the oscillator block 12 via a first data coupler 22.

More specifically, the first data unit 18 is communicatively connected with a second data unit 23 of oscillator block 12 via the first data coupler 22.

The oscillator block 12 comprises the second data unit 23, an HF control unit 24, an HF unit 25 and a grid unit 26.

The grid unit 26 is supplied with grid voltage from grid block 14. The grid voltage is the usual alternating grid voltage depending on the country in which the HF generator 10 is operated-for example a sinusoidal alternating voltage having an effective value of 230 V between phase conductor and neutral conductor and a grid frequency of 50 Hz. The grid unit 26 can comprise a power factor correction unit 27 with which the power factor of the oscillator block 12 can be increased and thus disturbing harmonics can be reduced for the grid. The power factor correction unit 27 is controlled by means of the HF control unit 24.

The grid unit 26 can be configured to rectify the grid voltage and to forward the produced direct voltage to the HF unit 25. The created direct voltage can be higher or lower than the grid voltage of the grid block. The grid unit 26 can have, for example, a boost converter-that can also be denoted as step-up converter-for converting the rectified grid voltage.

In the HF unit 25 an oscillator circuit is provided with which a high frequency voltage signal is produced from the rectified stepped-up direct voltage. HF currents are produced from the HF voltage signal, for example by means of a power amplifier operating preferably in the switching mode, with which the medical instruments are supplied. The HF unit 25 is thereby controlled by the HF control unit 24.

The created HF currents of the HF unit 25 are transmitted from the oscillator block 12 to the output block 11 via the first power coupler 28. The first power coupler 28 can thereby serve as part of the power amplifier for producing the HF currents.

The insulation concept of the HF generator 10 according to the invention provides that the first power coupler 28 and the first data coupler 22 comprise a considerably higher insulation voltage compared to the remaining data and power couplers of the HF generator 10, by means of which the dielectric strength of a component is defined. This results in that the output block 11 can be effectively protected against a breakthrough of voltage peaks of grid block 14 to the output block 11 via oscillator block 12. For example, the insulation voltage of the first data coupler and the first power coupler can be two times the insulation voltage of the remaining data and power couplers. Due to the in-creased insulation of the output block 11 from the oscillator block 12, it is possible to reduce the number of insulation locations between the blocks and thereby simplify the total architecture of the HF generator 10.

The communication block 13 comprises a second power coupler 29 and a third power coupler 30. The second power coupler 29 and the third power coupler 30 comprise a lower insulation voltage than the first power coupler 28. For example, the sum of the insulation voltages of the second power coupler 29 and the third power coupler 30 correspond to the insulation voltage of first power coupler 28. The second and third power couplers 29, 30 serve to supply the units comprised by the connected blocks, that is the communication block 13 and the output block 11, with an operating voltage.

The communication block 13 comprises an operation control unit 31, which is connected with a communication interface 32, that comprises a multiplicity of operation and indication interfaces 33a to 33g. The operation and indication interfaces 33a to 33g can comprise, for example, a speaker interface 33a, a pedal interface 33b, additional assistive input interfaces 33c, 33d, 33e, 33f (for example a Universal Serial Bus (USB) or other communication buses) as well as a display interface 33g. The operation and indication interfaces 33a to 33g allow the operator to input operation parameters for the HF generator 10, for example via an operation unit, such as a touch screen, as well as to output (current) sensor data, operation parameters or the like. This can be carried out via an indication unit 34, for example, that comprises a display. The control of the communication block 13 is carried out using the operation control unit 31.

The operation control unit 31 is connected with the second data unit 23 via a second data coupler 35. Also, the second data coupler 35 comprises a lower insulation voltage than the first data coupler 22. The first data coupler 22 and the second data coupler 35 can be configured as optocouplers, for example.

Between the oscillator block 12 and grid block 14 at least a fourth power coupler 36 can be arranged in addition. The fourth power coupler 36 can have a considerably lower insulation voltage than the other power couplers 28, 29, 30. During operation no danger exists that the patient, the operator, or the surgeon gets in contact with the oscillator block 12, whereby remarkably lower requirements can be made to the dielectric strength between grid block 14 and oscillator block 12. If a fourth power coupler 36 is provided, it can serve to supply the units comprised by oscillator block 12 with operating voltage. Alternatively, the units of oscillator block 12 can also be directly supplied by grid block 14.

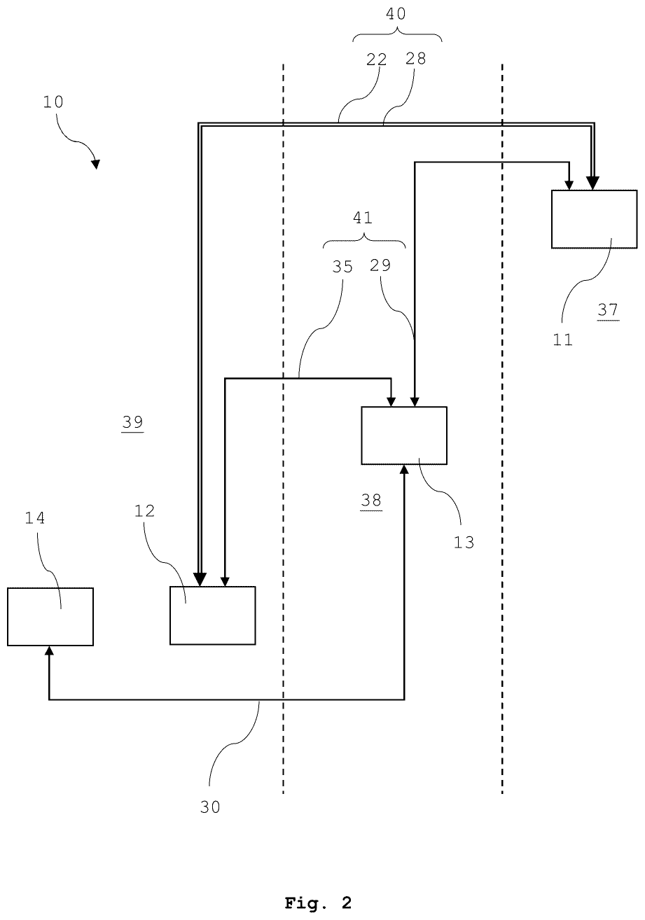

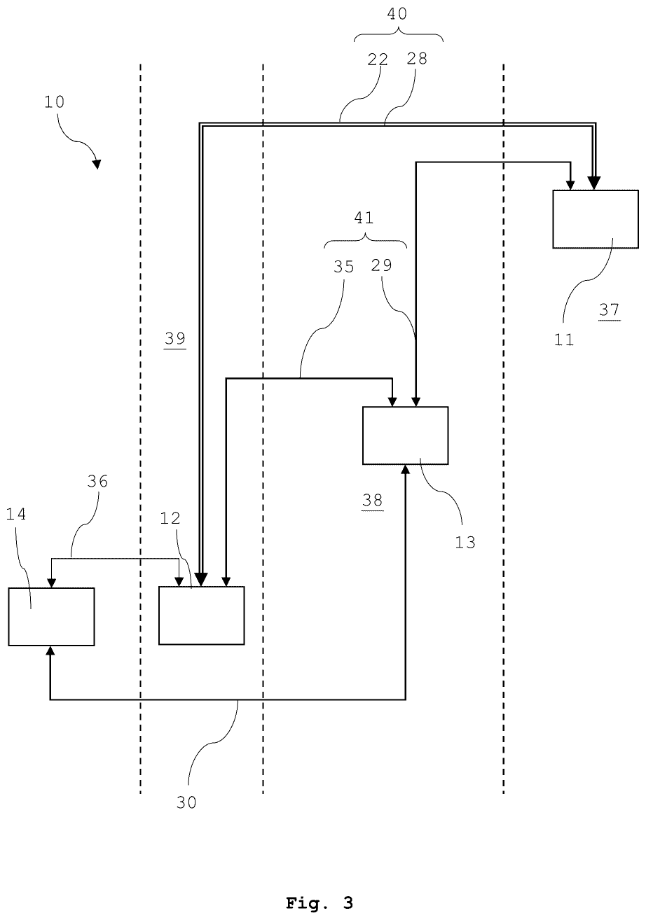

Between the individual blocks 11, 12, 13, and 14 of HF generator 10 isolation paths are defined via first power coupler 28, second power coupler 29, third power coupler 30, fourth power coupler 36, first data coupler 22 and second data coupler 35, which are explained in the following in more detail based on FIG. 2 and FIG. 3.

FIG. 2 and FIG. 3 illustrate the insulation scheme of the HF generator 10 according to the invention. In FIGS. 2 and 3 the vertical distance between the blocks that means in direction of the height-represent the insulation voltages of the individual power and data couplers between one another.

In both examples depicted in FIG. 2 and in FIG. 3 the insulation voltages of first power coupler 28 and first data coupler 22 are twice the insulation voltages of second data coupler 35 and second power coupler 29.

The HF generator 10 is divided in multiple insulation zones, namely a high insulation zone 37, an intermediate insulation zone 38 and a low insulation zone 39.

In the example shown in FIG. 2 the oscillator block 12 and the grid block 14 are both arranged in the low insulation zone 39. This means that between the two blocks no or only a negligible low insulation voltage is provided. The output block 11 is assigned to the high insulation zone 37.

The communication block 13 is assigned to the intermediate insulation zone 38 on the contrary.

Between the output block 11 and the oscillator block 12 a first insulation path 40 is formed in which first data coupler 22 and first power coupler 28 are arranged parallel to one another.

Between the output block 11 and oscillator block 12 in addition a second insulation path 41 via second power coupler 29, communication block 13 and second data coupler 35 (in series). The first insulation path 40 and the second insulation path 41 are in turn arranged parallel to each other. Moreover, communication block 13 is insulation protected in relation to grid block 14 by means of third power coupler 30.

FIG. 3 illustrates another insulation scheme for the HF generator 10 according to the invention. With reference to the already introduced reference signs the above explanations apply accordingly. The example according to FIG. 3 distinguishes from the example according to FIG. 2 in that a fourth power coupler 36 is arranged between grid block 14 and oscillator block 12.

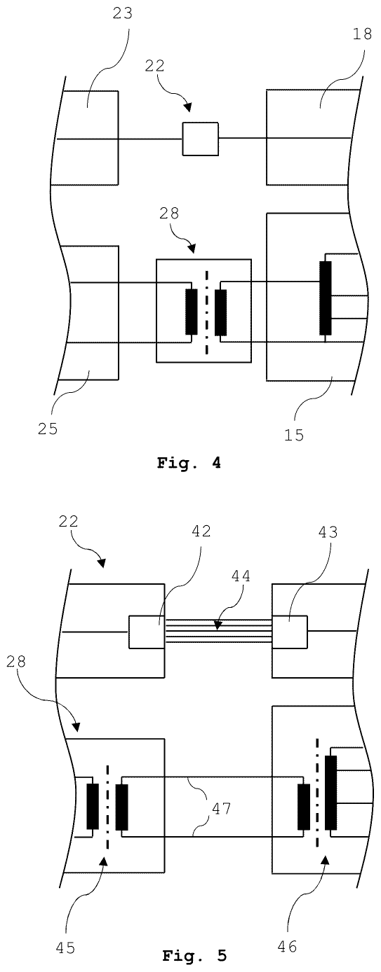

FIG. 4 shows a detailed illustration of an example of first data coupler 22 and first power coupler 28. In this example first data coupler 22 is configured as optocoupler. The first power coupler 28 is configured as transformer in the illustrated example. The insulation voltage of the transformer describes its ability to withstand a voltage breakthrough between its primary winding and its secondary winding. For example, the transformer is dimensioned to have an insulation voltage of 12 kV. This means that a voltage difference between the primary side and the secondary side of the transformer can have an amount up to 12 kV without resulting in a short circuit between primary side and secondary side of the transformer. Primary side and secondary side are thus reliably electrically isolated from each other. On the secondary side an autotransformer for realizing one or more taps is provided in the example illustrated in FIG. 4, from which the medical instruments can be supplied.

FIG. 5 shows a detailed illustration of an alternative example for the first data coupler 22 and the first power coupler 28. In this example first data coupler 22 comprises an oscillator block side optocoupler 42 and an output block side optocoupler 43, which are connected with each other via multiple optical connection lines 44.

In the example shown in FIG. 4, the insulation voltage of first data coupler 22 results from the sum of the insulation voltages of the oscillator block side optocoupler 42 and the output block side optocoupler 43. For example, the oscillator block side optocoupler 42 can have an insulation voltage of 6 kV and the output block side optocoupler 43 can also have an insulation voltage of 6 kV. In total the first data coupler 22 then comprises an insulation voltage of 12 kV.

The first power coupler 28 comprises an oscillator block side transformer 45 and an output block side transformer 46. The secondary side of the oscillator block side transformer 45 is connected with the primary side of the out put block side transformer 46 via transmission line 47.

In this example, the oscillator block side transformer 45 and the output block side transformer 46 are dimensioned for an insulation voltage in each case which sum up due to the series connection of the two transformers. For example, the insulation voltage of the two transformers has an amount of 6 kV, so that first power coupler 28 comprises an insulation voltage of 12 kV in total. In this embodiment it is advantageous if the two transmission transformers 45, 46 have parasitic capacities between the respective primary winding and the respective secondary winding coinciding with one another respectively. This is the case although the two transformers 45, 46 cannot be identical in construction by definition due to the required voltage transmission or reduction ratio. Resulting from the coinciding parasitic capacities, however, a voltage peak between the primary side of transformer 45 and the secondary side of transformer 46 is divided equally on the two transformers 45, 46. In doing so, a serial voltage breakthrough through the power coupler 28 is avoided. As necessary one or more capacitors can be switched in parallel to the parasitic capacities in order to create the indicated voltage balance.

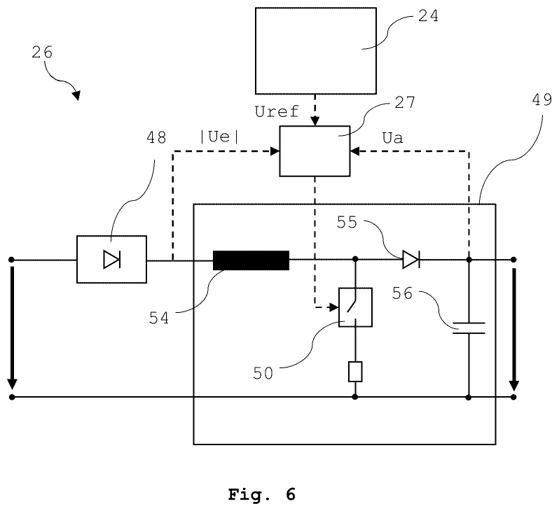

FIG. 6 illustrates the grid unit 26 of oscillator block 12. In the example shown in FIG. 6 the grid unit 26 comprises a rectifier 48 that converts a grid voltage on an input side into a direct voltage. The grid unit 26 additionally comprises a boost converter 49 with which the created direct voltage can be boosted.

The boost converter 49 comprises a switch 50, an inductor 54, a diode 55 and a capacitor 56. Due to the inductance of the inductor 54 the current flow remains, if switch 50 is opened. The voltage on the output side end increases therefore rapidly until the voltage applied to capacitor 56 is exceeded and therefore diode 55 is conductive.

Thus, in the first instance the current continues to flow unmodified and further charges capacitor 56. The magnetic field of the inductor is thereby reduced and outputs its energy in that current is driven via diode 55 into capacitor 56. The capacity of capacitor 56 is dimensioned so that the output voltage is approximately constant during an operation cycle.

The switch 50 is controlled by power factor correction unit 27. The power factor correction unit 27 receives the absolute value of the rectified grid voltage (the input voltage Ue) and the output voltage (Ua) of the boost converter 49 as well as a reference voltage Uref from HF control unit 24. In the power factor correction unit 27 the difference between output voltage Ua and the reference voltage is multiplied with the absolute value of the input voltage Ue in order to calculate a desired current with which switch 50 is controlled. The power factor can thus be set to a value close to 1.

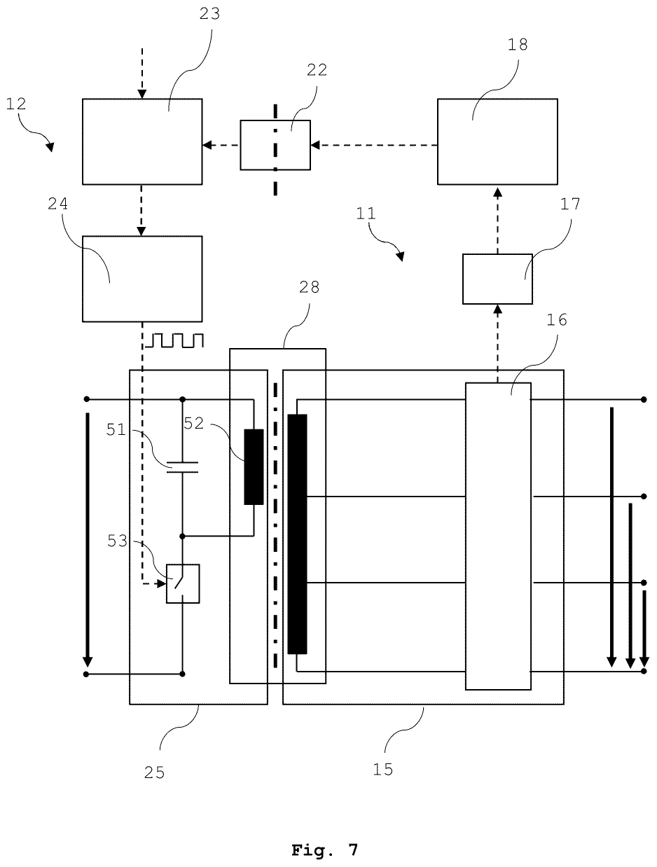

FIG. 7 shows a detailed illustration of an HF unit 25 having the first power coupler 28 and the distribution unit 15 of output block 11. The HF unit 25 comprises an oscillator circuit comprising a capacitor 51, an inductor 52 and a switch 53. The switch 53 is controlled by HF unit 24.

The inductor 52 of HF unit 25 can be already one side of first power coupler 28, which is configured as transformer, for example. In FIG. 7, inductor 52 is the primary winding of the transformer of first power coupler 28. On the secondary side of first power coupler 28 the distribution unit 15 is connected. The secondary winding of the transformer can thereby also be used to tap different HF currents having different voltage levels. In the distribution unit 15 a sensor unit 16 is provided by means of which currents, voltages, complex resistances and the like can be detected that are applied on the output side. The sensor unit 16 is connected with the preprocessing unit 17 that preprocesses the sensor data and forwards the sensor data via the first data unit by means of the first data coupler to the control unit 24.

The invention refers to an HF generator 10 for the supply of at least one medical instrument, particularly of at least one HF surgical instrument that is configured, for example, for cutting, for coagulating as well as for achieving additional tissue effects on biological tissue on a human or animal patient as desired. The HF generator 10 according to the invention comprises an output block 11, an oscillator block 12, a communication block 13 and a grid block 14. The output block 11 is configured to supply the one or the multiple medical instruments with an HF current. The oscillator block 12 is configured to supply the output block 11 via a first power coupler 28 with the HF current. In addition, the oscillator block 12 is connected with the output block 11 via a first data coupler 22. The communication block 13 is connected with the output block 11 via a second power coupler 29 and with the oscillator block 12 via a second data coupler 35. A particularity of the HF generator according to the invention is that the first power coupler 28 and the first data coupler 22 comprise a higher insulation voltage than the remaining power couplers 29, 30 and the data coupler 35, whereby the output block 11 is protected in relation to oscillator block 12 by means of only two couplers, which are provided with measures for increasing their insulation voltage, while providing similar functionality and equal dielectric strength.

1. A high frequency (HF) generator for supply of one or more medical instruments, the HF generator comprising:
an output block that is configured to supply the one or more medical instruments with an HF current;
an oscillator block that is configured to supply the output block via a first power coupler with the HF current, wherein the oscillator block is connected with the output block via a first data coupler;
a communication block that is connected with the output block via a second power coupler and that is connected with the oscillator block via a second data coupler; and
a grid block that is connected with the oscillator block and via a third power coupler with the communication block in supplying manner,
wherein the first power coupler comprises a higher insulation voltage than the second power coupler and the third power couplers.

2. The HF generator according to claim 1, wherein the first data coupler comprises a higher insulation voltage than the second data coupler.

3. The HF generator according to claim 1, wherein a first insulation path is formed between the output block and the oscillator block, and the first power coupler and the first data coupler are arranged parallel to one another.

4. The HF generator according to claim 1, wherein a second insulation path is formed between the output block and the oscillator block, and the second power coupler and the second data coupler are arranged in series to one another.

5. The HF generator according to claim 1, wherein an insulation strength of the first power coupler and an insulation strength of the first data coupler are, respectively, at least equal to or higher than an insulation strength of the second power coupler and an insulation strength of the second data coupler in total.

6. The HF generator according to claim 1, wherein the first and the second data coupler are configured as inductive or capacitive data couplers or as optocouplers.

7. The HF generator according to claim 1, wherein the oscillator block comprises an HF unit, an HF control unit, a grid unit and a second data unit, wherein the HF control unit is configured to control the HF unit, such that the HF unit creates HF currents having different parameters, wherein the parameters include at least one of: different current values, voltage values, waveforms, crest factors, clockings, and modes.

8. The HF generator according to claim 7, wherein the grid unit comprises a power factor correction unit, wherein the HF control unit is configured to adapt a power factor by means of the power factor correction unit.

9. The HF generator according to claim 1, wherein the output block comprises a distribution unit that is configured to distribute the HF current received via the first power coupler to the one or the one or more medical instruments.

10. The HF generator according to claim 9, wherein the distribution unit comprises at least one sensor unit that is configured for detection of the HF currents as sensor data in the output block, wherein the sensor data comprises at least one of: current, voltage, apparent power, active power, reactive power measurement values, a complex impedance of a tissue, changes of the complex impedance of a tissue, and/or, a linearity value of the complex impedance of a tissue.

11. The HF generator according to claim 10, wherein the output block comprises a first data unit that is communicatively connected with the at least one sensor unit and that is configured for distributing and buffering the sensor data.

12. The HF generator according to claim 11, wherein the first data unit is communicatively connected with a second data unit via the first data coupler.

13. The HF generator according to claim 7, wherein the communication block comprises an operation control unit and one or more operation and indication interfaces adapted to provide a user an input for one or more input parameters for the HF control unit and which are connected with the operation control unit.

14. The HF generator according claim 13, wherein the operation control unit is communicatively connected with the second data unit via the second data coupler.

15. The HF generator according to claim 1, wherein the output block, the oscillator block, and the communication block are assigned to different isolation zones in relation to the grid block, wherein the isolation zones have different isolation voltages, the isolations zones having a high insulation zone, an intermediate insulation zone, and a low insulation zone.

16. The HF generator according to claim 1, wherein the output block is protected by means of the second power coupler and the third power coupler in relation to the grid block.
